(19) United States
(12) Patent Application Publication
(10) Pub. No.: US 2005/0124891 A1
(43) Pub. Date: Jun. 9, 2005

(54) Ultrasonic diagnostic apparatus and driving method therefor

(76) Inventor: Shinichi Amemiya, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 11/008,314

(22) Filed: Dec. 9, 2004

(30) Foreign Application Priority Data
Dec. 9, 2003 (JP) 2003-410835

(51) Int. Cl. A61B 8/02

(52) U.S. Cl. 600/447

(57) ABSTRACT

An ultrasonic diagnostic apparatus for continuously transmitting ultrasonic signal toward a subject, continuously receiving signals reflected from the subject in response to the transmitted ultrasonic signals, and producing a tomographic image of a subject based on the received signals, comprises transmitting/receiving means for transmitting/receiving ultrasonic signals via a plurality of channels, and a sector probe having transducers connectable to the channels via switches, the transducers being in a number larger than the number of the channels and arranged in one direction, in which probe, ones among said transducers arranged in one direction that are spaced at intervals of a predetermined number of said transducers are connected to channels for transmitting ultrasonic signals.

### BACKGROUND OF THE INVENTION

The present invention relates to an ultrasonic diagnostic apparatus and a driving method therefor, and particularly to an ultrasonic diagnostic apparatus comprising a probe having a larger number of transducers than the number of the channels in transmitting/receiving means, for producing an image by a continuous-wave Doppler technique, and a driving method for such an apparatus.

Apparatuses for scanning a predefined region in a subject to be imaged by ultrasound to produce an image of the subject include the ultrasonic diagnostic apparatus. The ultrasonic diagnostic apparatus is attracting attention because it can conduct a scan and produce an image without distressing the subject to be imaged.

Regarding the ultrasonic diagnostic apparatus, the following techniques are known for determining the dynamics, i.e., velocity information, of blood flow: a continuous-wave Doppler (sometimes abbreviated as CWD hereinbelow) method and a pulse-wave Doppler (sometimes abbreviated as PWD hereinbelow) method.

The CWD method is useful for obtaining velocity data because it employs continuous waves in transmitting ultrasonic signals and it can accurately detect a relatively high flow rate. In the CWD method, a technique involving transmitting continuous waves deflected with respect to the subject is generally referred to as steerable CWD, in which continuous waves with a phase difference are transmitted from transmitting transducers into the subject and reflected waves are received from the subject.

When steerable CWD is implemented in an ultrasonic diagnostic apparatus comprising a probe in which a plurality of transducers are linearly arranged, and a transceiver section having channels connectable to respective transducers, for example, transducers in a continuous area in the center or on one side of the probe are used as the transmitting transducers. Moreover, transducers in a continuous area other than those for use in transmission are employed as receiving transducers. In this case, the number of transducers is larger than that of the channels.

As such a conventional ultrasonic apparatus, there is known an ultrasonic diagnostic apparatus for acquiring continuous-wave Doppler data that is capable of suitably acquiring the continuous-wave Doppler data by reducing received power loss even if there exists a relative angular variation between the ultrasonic probe and blood vessel, in which the ultrasonic probe has its axis of rotation aligned with a direction along which the transmitting and receiving elements are arranged (e.g., see Patent Document 1).

If transducers arranged in the probe in an area on either side of the probe are employed in transmission or reception as described in Patent Document 1, the apertures for transmission and reception become too small to attain sufficient resolution. For example, sensitivity to subtle counterflow in imaging blood flow becomes insufficient.

Moreover, the number of the channels for use in reception is limited, sometimes resulting in insufficient reception sensitivity.

[Patent Document 1] Japanese Patent Application Laid Open No. 2001-170052.

### SUMMARY OF THE INVENTION

Therefore, an object of the present invention is to provide an ultrasonic diagnostic apparatus and a driving method therefor for improving resolution of an image to be produced by widening an aperture area in transmitting ultrasonic signals, and improving reception sensitivity.

To attain the aforementioned object, an ultrasonic diagnostic apparatus of the present invention is for continuously transmitting ultrasonic signals toward a subject, continuously receiving signals reflected from the subject in response to the transmitted ultrasonic signals, and determining dynamics of blood flow in the subject based on the received signals, and the apparatus comprises: transmitting/receiving means for continuously transmitting/receiving ultrasonic signals via a plurality of channels; and a sector probe having transducers connectable to the channels via switches, the transducers being in a number larger than the number of the channels and arranged in one direction, in which probe, ones among said transducers arranged in one direction that are spaced at intervals of a predetermined number of said transducers are connected to channels for transmitting ultrasonic signals.

According to the ultrasonic diagnostic apparatus of the present invention, ones among said transducers arranged in one direction that are spaced at intervals of a predetermined number of said transducers are connected to channels for transmitting ultrasonic signals.

To attain the aforementioned object, a driving method of the present invention is for an ultrasonic diagnostic apparatus comprising transmitting/receiving means for continuously transmitting/receiving ultrasonic signals via channels, and a sector probe having transducers connectable to the channels via switches, the transducers being in a number larger than the number of the channels and arranged in one direction, and the method comprises the steps of: continuously transmitting ultrasonic signals toward a subject while connecting transducers in a predefined area to said channels, and continuously receiving reflected signals while connecting transducers in the other area to said channels; and producing an image of the subject based on the received signals, wherein the transmitting/receiving step comprises selecting a plurality of transducers in the predefined area spaced at intervals of a predetermined number of transducers, and transmitting the ultrasonic signals while connecting the selected transducers to the channels.

According to the driving method of the present invention, ultrasonic signals are continuously transmitted toward a subject while connecting transducers in a predefined area to the channels, and reflected signals are continuously received while connecting transducers in the other area to the channels. At that time, a plurality of transducers in the predefined area are selected at intervals of a predetermined number of transducers, and the ultrasonic signals are transmitted while connecting the selected transducers to the channels. Then, an image of the subject is produced based on the received signals.

According to the ultrasonic diagnostic apparatus of the present invention, resolution of an image to be produced is improved by widening the aperture area in transmitting ultrasonic vibration, and reception sensitivity is also improved.

According to the driving method of the present invention, resolution of an image to be produced is improved by widening an aperture area in transmitting ultrasonic vibration, and reception sensitivity is also improved.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram schematically showing an ultrasonic diagnostic apparatus 1 in accordance with one embodiment of the present invention.

FIG. 2 is a schematic enlarged diagram showing an example of the ultrasonic diagnostic apparatus 1 of the present invention.

FIG. 3 is a schematic diagram generally showing an example of connections between a transceiver section and transducers in the ultrasonic diagnostic apparatus 1 shown in FIG. 2.

FIG. 4 is a flow chart for explaining an operation of the ultrasonic diagnostic apparatus 1 shown in FIG. 1.

FIG. 5 is a flow chart for explaining an operation of the ultrasonic diagnostic apparatus 1 shown in FIG. 3.

FIG. 6 is a schematic diagram generally showing another example of connections between the transceiver section and transducers in the ultrasonic diagnostic apparatus 1 shown in FIG. 2.

FIG. 7 is a schematic enlarged diagram showing an example of the ultrasonic diagnostic apparatus 1 of the present invention.

FIG. 8 is a schematic diagram generally showing an example of connections between the transceiver section and transducers in the ultrasonic diagnostic apparatus 1 shown in FIG. 6.

FIG. 9 is a schematic diagram generally showing another example of connections between the transceiver section and transducers in the ultrasonic diagnostic apparatus 1 shown in FIG. 6.

### DETAILED DESCRIPTION OF THE INVENTION

The best mode for carrying out the present invention will now be described with reference to the accompanying drawings.

### First Embodiment

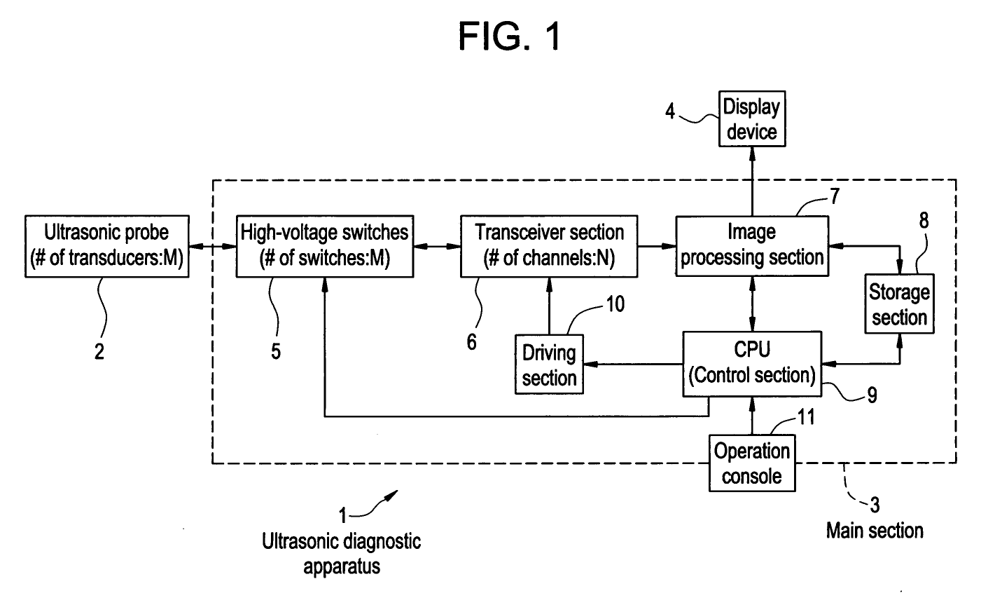

FIG. 1 is a schematic block diagram generally showing an ultrasonic diagnostic apparatus 1 in accordance with the present embodiment.

The ultrasonic diagnostic apparatus 1 in accordance with the present embodiment comprises an ultrasonic probe 2, a main section 3, and a display device 4. The ultrasonic probe 2 and main section 3 are connected to each other via a probe cable, which is not shown. In ultrasonic imaging, a physician, for example, is the user conducting a scan with the ultrasonic diagnostic apparatus 1. In imaging, the ultrasonic probe 2 is held by the user and is put against the subject.

The ultrasonic probe 2 is connected to a transceiver section 6 via high-voltage switches 5 in the main section 3, which will be discussed later. The ultrasonic probe 2 has a sector array in which M transducers are arranged in one direction, for example.

The ultrasonic probe 2 converts electric signals supplied from the transceiver section 6 via the high-voltage switches 5 into ultrasound by the transducers and transmits them to the subject. The ultrasonic probe 2 also converts reflected waves from the subject into electric signals by the transducers and outputs them to the transceiver section 6 via the high-voltage switches 5. The ultrasound transmitted by the transducers forming an acoustic line generates echoes corresponding to the difference in acoustic impedance of the internal tissue of the subject during travel inside the subject. These echoes are received by the ultrasonic transducer array and converted into electric signals. As used herein, the term ultrasonic signals refer to both electric signals and ultrasound converted by the transducers.

The ultrasonic probe 2 used in the present embodiment is a sector probe for scanning a wide field of view. One embodiment of the probe of the present invention corresponds to the ultrasonic probe 2.

The main section 3 comprises the high-voltage switches 5, the transceiver section 6, an image processing section 7, a storage section 8, a CPU (control section) 9, a driving section 10, and an operation console 11. The main section 3 produces several kinds of ultrasonic images of the subject based on electric signals (echo signals) supplied via the probe cable.

In the present embodiment, the main section 3 produces an image of the subject based on offset between transmitted and received waves, i.e., a Doppler pattern, for example. The main section 3 will now be described hereinbelow.

The high-voltage switches 5 are connected to the transceiver section 6, ultrasonic probe 2, and control section 9. The high-voltage switches 5 consist of, for example, M number of switches. The high-voltage switches 5 are turned on/off based on instructions from the control section 9 to connect the transceiver section 6 and ultrasonic probe 2. One embodiment of the switches of the present invention corresponds to the high-voltage switches 5.

The transceiver section 6 is connected to the high-voltage switches 5, image processing section 7, and driving section 10. The transceiver section 6 is a port for signal transmission and reception. The transceiver section 6 has, for example, N channels. The number of the channels N is smaller than the number of transducers M in the ultrasonic probe 2. The transceiver section 6 sends driving signals supplied from the driving section 10 for driving the ultrasonic probe 2 to the ultrasonic probe 2 via the probe cable. Moreover, the transceiver section 6 sends ultrasonic signals received from the ultrasonic probe 2 via the probe cable to the image processing section 7. One embodiment of the transmitting/receiving means of the present invention corresponds to the transceiver section 6.

The control section 9 is connected to the driving section 10, image processing section 7, storage section 8, and operation console 11. The control section 9 outputs a command signal for causing the ultrasonic probe 2 to transmit ultrasound for imaging to the driving section 10. The control section 9 also outputs a command signal for displaying a tomographic image, for example, according to instructions represented by an operation signal from the operation console 11, to the image processing section 7. Moreover, the control section 9 controls storage of image data in the storage section 8 based on instructions from the operation console 11.

The driving section 10 is implemented using an electric/electronic circuit, for example. The driving section 10 generates a driving signal for driving the ultrasonic probe 2 to form an acoustic line in response to a command signal from the control section 9, and sends the generated driving signal to the transceiver section 6.

The image processing section 7 produces an image of the subject based on ultrasonic signals sent by the transceiver section 6. The image processing section 7 also causes the display device 4 to display an image produced in response to instructions from the control section 9. Moreover, the image processing section 7 sends image data to the storage section 8 for storage. The image processing section 7 is comprised of a program and the like.

The storage section 8 may include several kinds of storage devices such as a semiconductor memory and a hard disk drive. The storage section 8 stores image data sent from the image processing section 7. The storage section 8 also stores a program for operating the ultrasonic diagnostic apparatus 1, acoustic lines used in the program, and several kinds of parameters such as distance to the subject to be imaged.

The operation console 11 is an apparatus for accepting an operation by the operator for operating the ultrasonic diagnostic apparatus 1. The operation console 11 is comprised of input sections such as a keyboard and switches.

The display device 4 displays an image produced in the main section 3 and other imaging data. The display device 4 is comprised of a CRT or a crystal liquid display panel, for example.

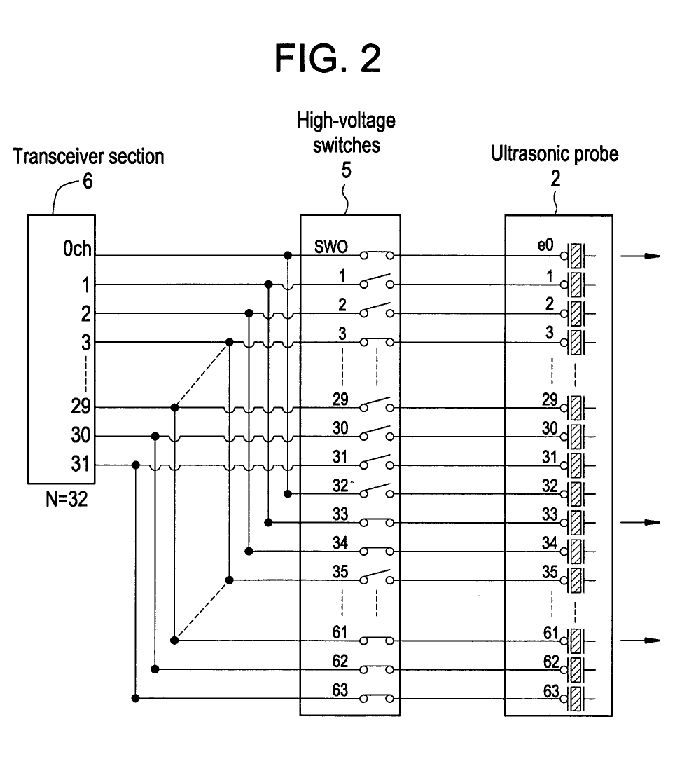

FIG. 2 is an explanatory diagram showing connections between the ultrasonic probe 2 and transceiver section 6 in accordance with the present invention.

While the number of transmission and reception channels in the ultrasonic diagnostic apparatus is generally more than 32, the present embodiment addresses a case in which the number of the channels in the transceiver section 6 is N=32 for simplification. The number of the high-voltage switches 5 and the number of transducers in the ultrasonic probe 2 are M=63 here. The elements in the ultrasonic probe 2 are arranged in one direction.

Representing the channel indices as N=0-31, an N-th channel is connectable to an N-th switch and an (N+32)-th switch in parallel. Moreover, zero-th element e0-63^{rd} element e63 in the ultrasonic probe 2 are connectable to certain channels via zero-th switch SWO-63^{rd} switch SW63.

The steerable CWD method conducts transmission and reception in a continuous manner. Therefore, each channel conducts either transmission or reception. A channel for transmitting an ultrasonic signal to an element will be sometimes referred to as a transmitting channel, and a channel for receiving an ultrasonic signal reflected at the subject from an element as a receiving channel hereinbelow.

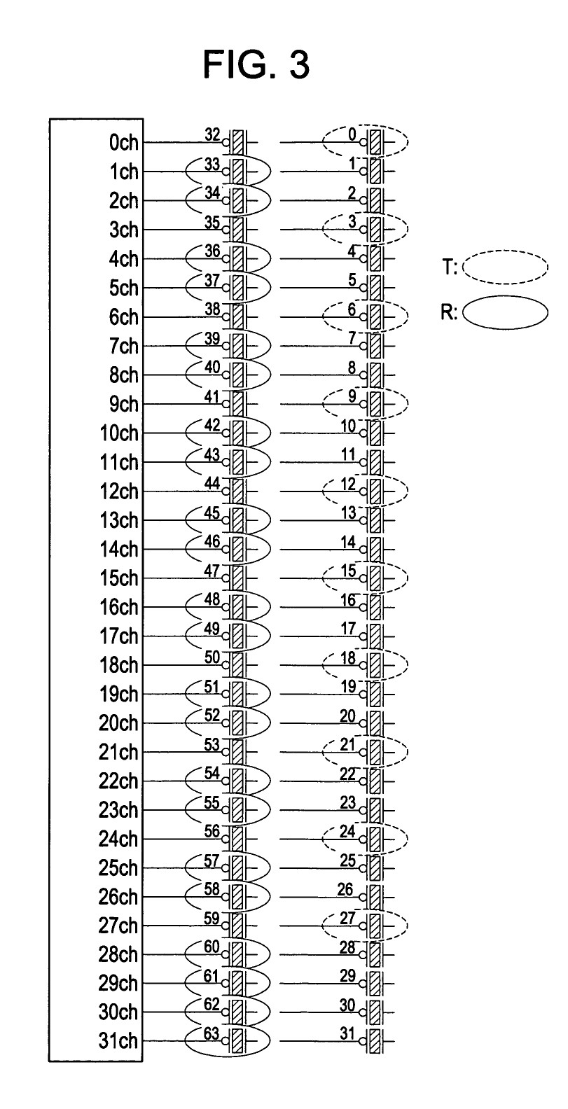

FIG. 3 is a schematic diagram generally showing an example of connections between channels and elements. It should be noted that the switches SWO-SW63 shown in FIG. 2 are omitted in FIG. 3. A transmitting element T connected to a transmitting channel via a high-voltage switch 5 is surrounded by a dashed line, and a receiving element R connected to a receiving channel is surrounded by a solid line.

As shown in FIG. 3, in zero-th through 27^{th} elements e0-e27, every third element is connected to a corresponding channel via a high-voltage switch 5 (not shown). The channels corresponding to the selected elements output transmitted signals, and are regarded as transmitting channels. Moreover, an element at one end through an element at the other end that are connected to these channels, i.e., the zero-th through 27^{th} element e0-e27, are together regarded as a transmission area.

On the other hand, 33^{rd} through 63^{rd} elements e33-e63 outside the transmission area are connected to all channels other than those connected to the aforementioned elements. The channels connected to the 33^{rd} through 63^{rd} elements e33-e63 input received signals, and are regarded as receiving channels. Moreover, 33^{rd} through 63^{rd} elements e33-e63 are together regarded as a reception area.

Now an operation of the ultrasonic diagnostic apparatus 1 in accordance with the present invention will be described with reference to the drawings.

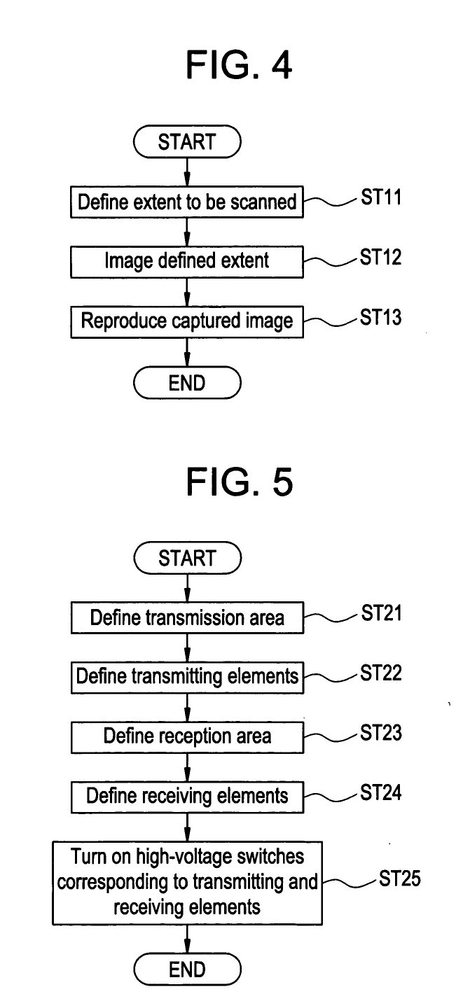

FIG. 4 is a flow chart showing an operation of the ultrasonic diagnostic apparatus 1 in accordance with the present embodiment.

First, an extent to be scanned using the ultrasonic probe 2 is defined (ST11). The user positions the ultrasonic probe 2 at a certain location in the subject. At that time, depth dimension, azimuthal dimension and thickness dimension of the extent to be scanned are determined depending upon the frequency or scan shape so that the extent to be scanned corresponds to an object to be detected. The extent to be scanned is desirably defined to contain at least the object to be detected.

Next, the region defined at Step ST11 in the subject rested at a predetermined position is imaged (ST12).

The user puts the ultrasonic probe 2 against a scanned position. The user also operates the operation console 11 to supply a command to the driving section 10 via the control section 9.

The driving section 10, in response to a command signal from the control section 9, generates a driving signal for continuously forming a predetermined acoustic line from combined wavefronts of ultrasound from the ultrasonic transducer array in the ultrasonic probe 2, and outputs it to the ultrasonic probe 2 via transmitting channels established in the transceiver section 6.

The driving section 10 continuously forms a plurality of acoustic lines in one plane (scan plane) in the predefined region, and the ultrasonic probe 2 continuously scans the scan plane by the plurality of acoustic lines. The ultrasonic probe 2 also continuously receives ultrasonic signals coming from the inside of the subject. Such transmission and reception are simultaneously conducted. This step corresponds to one embodiment of the transmitting/receiving step of the present invention.

The step of outputting a signal to the ultrasonic probe 2 at Step ST12 corresponds to the driving method for the ultrasonic diagnostic apparatus of the present invention, details of which will be discussed later.

The transceiver section 6 sends ultrasonic signals continuously received by the ultrasonic probe 2 via the probe cable to the image processing section 7. Based on the ultrasonic signals sent from the transceiver section 6 to the image processing section 7, the image processing section 7 continuously compares transmitted and received waves to find an offset therebetween, and analyzes the difference in wavelength between transmitted and received waves, i.e., analyzes the changed frequency component, to generate a Doppler pattern such as that displayed as a spectrum. This step corresponds to one embodiment of the image producing step of the present invention.

The captured image is stored in the storage section 8 based on instructions by the control section 9.

The captured image stored in the storage section 8 is then reproduced at the display device 4 (ST13).

At Step ST12, the control section 9 selects transmitting elements to be connected to the transmitting channels and receiving elements to be connected to the receiving channels by the high-voltage switches 5, and turns on the high-voltage switches 5 corresponding to the selected transmitting and receiving elements. At that time, the control section 9 selects the plurality of elements to be connected to the transmitting channels separated by a predetermined number of elements in a predefined area, and turns on the high-voltage switches 5.

The step of selecting elements to be connected to transmitting and receiving channels will now be described in detail with reference to the accompanying drawings.

FIG. 5 is a flow chart showing an example of the step of selecting elements to be connected to transmitting and receiving channels.

The control section 9 first defines a transmission area of elements with respect to their arrangement direction (ST21). The transmission area is desirably defined to contain about ¼-½ of the total number of elements. Below ¼, the aperture in the transmission area becomes small. As a result, the angle of ultrasonic signal emission diverges to degenerate resolution. On the other hand, above ½, the aperture in the reception area becomes small and the S/N ratio is reduced. For example, in the ultrasonic probe 2 having 64 elements arranged as shown in FIG. 3, zero-th element e0-27^{th} element e27 are defined as the transmission area.

Next, in the thus-defined transmission area, elements to transmit ultrasonic signals are selected (ST22).

The control section 9 selects elements to be connected to transmitting channels in the defined transmission area. For example, as shown in FIG. 3, every third element is selected, the predetermined interval being two in this case. The selected elements are defined as the transmitting elements.

Next, a reception area is defined (ST23) .

The control section 9 defines the reception area separated by a predetermined interval from the transmission area. For example, as shown in FIG. 3, the reception area is defined to contain 33^{rd} through 63^{rd} elements e33-e63 separated by 28^{th} through 32^{nd} elements e28-e32 from the transmission area.

Next, in the reception area, elements that are not connected to the transmitting channels are selected (ST24) .

The control section 9 selects elements corresponding to channels that are not connected to elements in the reception area, as exemplarily shown in FIG. 3, and defines them as the receiving channels and receiving elements.

Once the transmitting and receiving elements have been selected as described above, the control section 9 turns on the high-voltage switches 5 corresponding to the selected transmitting channels and those corresponding to the receiving channels (ST25).

Consequently, the channels continuously transmit/receive ultrasonic signals by different elements in the ultrasonic probe 2 to/from the transceiver section 6. The image processing section 7 then produces an image based on thus-transmitted/received data.

(Variation)

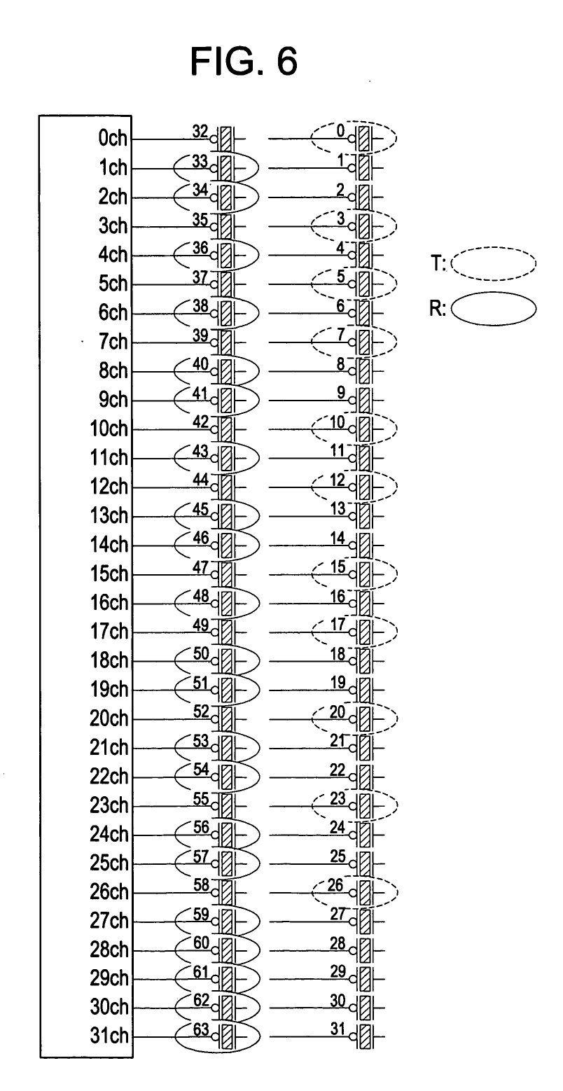

FIG. 6 is a schematic diagram generally showing another example of connections between channels and elements. As shown in FIG. 6, in zero-th through 26^{th} elements e0-e26, every third or every second element is randomly connected to a channel via the high-voltage switch 5 to form the transmission area.

On the other hand, in 33^{rd} through 63^{rd} elements e33-e63 outside the transmission area, elements are connected to all channels other than those connected to the transmitting elements to form the reception area. Moreover, channels corresponding to 27^{th} through 32^{nd} elements e27-e32 are connected to the counterpart of connectable elements, i.e., zero-th element e0 and 59^{th}-63^{rd} elements e59-e63. Accordingly, the high-voltage switches 52 corresponding to the 27^{th} through 32^{nd} elements e27-e32 are turned off. As a result, isolation can be established between the transmission and reception areas.

According to the first embodiment of the present invention, elements arranged in one direction are divided into a transmission area and a reception area, and in the transmission area, elements to be connected to transmitting channels are defined to be spaced at predetermined intervals. As a result, the transmission area is enlarged to widen the aperture, thus improving resolution of an image to be produced.

Moreover, since all channels other than transmitting channels are defined as receiving channels, and each receiving channel is connected to some element, reception can be achieved using many channels. As a result, reception sensitivity is improved, and the signal-to-noise ratio (sometimes abbreviated as the S/N ratio) is improved.

Furthermore, by randomly defining elements to be connected to transmitting channels as in Variation 1, grating lobes can be reduced. The CWD method generally conducts transmission and reception using ultrasound of low frequency. Therefore, grating lobes are prevented as compared with a B-mode technique even if every third element is connected to a transmitting channel.

While in the aforementioned embodiment, the transmission area is defined beforehand and transmitting elements are selected within the defined area, the transmitting elements may be selected without defining the transmission area.

### Second Embodiment

Now a second embodiment will be described. Similar portions to those in the first embodiment are designated by similar reference symbols and description thereof will be omitted.

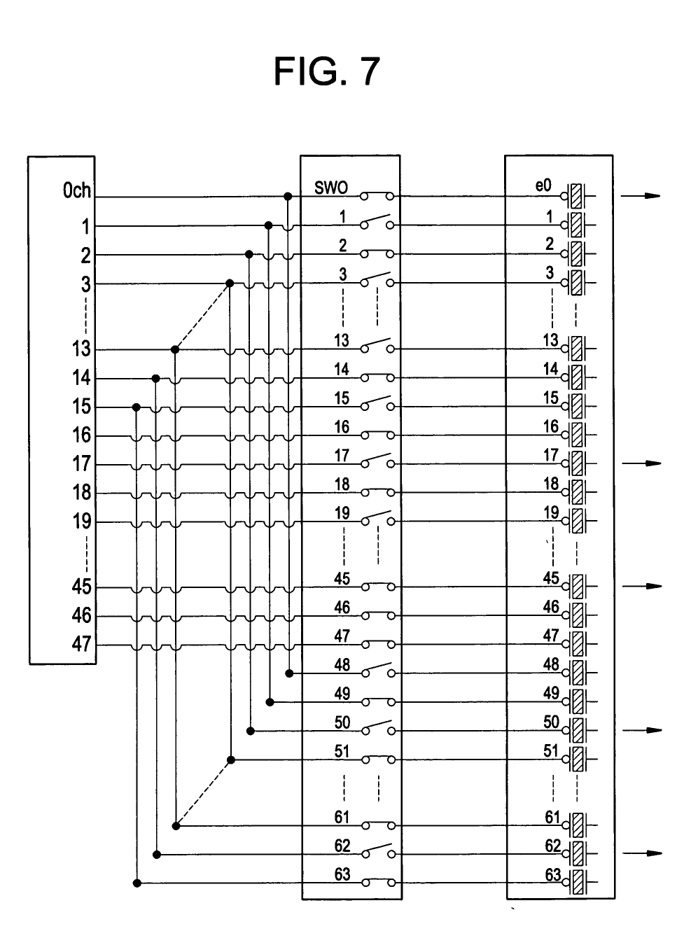

FIG. 7 is an explanatory diagram showing another example of connections between the ultrasonic probe 2 and transceiver section 6 in accordance with the present invention. For simplification, the number of the channels in the transceiver section 6 is exemplified as N=48 in the present embodiment.

As shown in FIG. 7, when the number of the channels in the transceiver section 6 is N=0-47, an N-th channel (N≦15) is connectable to an N-th switch and an (N+48)-th switch, and an N-th channel (16≦N≦47) is connectable to an N-th switch in parallel.

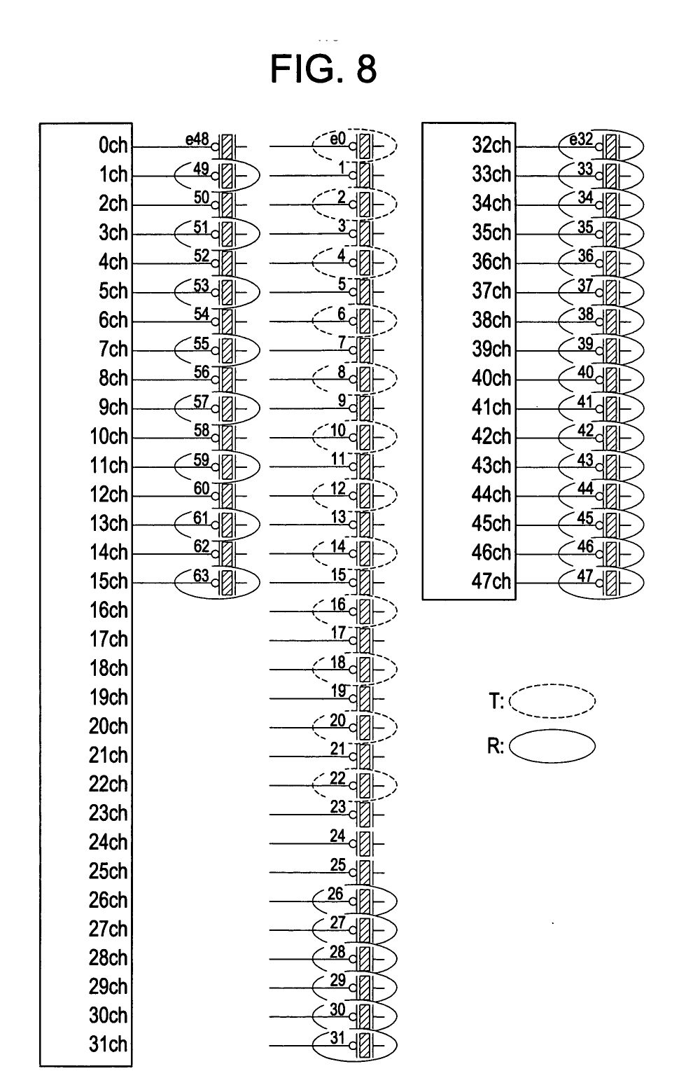

FIG. 8 is a schematic diagram generally showing an example of connections between the channels and elements shown in FIG. 7.

As shown in FIG. 8, in zero-th through 22^{nd} elements e0-e22, every second element is connected to a corresponding channel via a high-voltage switch 5 (not shown). The channels corresponding to the selected elements output transmitted signals, and are regarded as transmitting channels. Moreover, zero-th through 22 elements e0-e22 are together regarded as a transmission area.

On the other hand, 26^{th} through 63^{rd} elements e26-e63 outside the transmission area are connected to all channels other than those connected to the aforementioned elements. The connected channels input received signals, and are regarded as receiving channels. Moreover, 26^{th} through 63^{rd} elements e26-e63 are together regarded as a reception area.

Elements between the transmission and reception areas, i.e., 23^{rd} through 25^{th} elements e23-e25 are not used in either transmission or reception. In such a case, the high-voltage switches 5 corresponding to these elements e23-e25 are turned on, and are connected to channels that conduct neither transmission nor reception. As a result, the elements can be dampened to electrically and mechanically reduce cross-talk from transmission to reception.

Moreover, the 17^{th} element e17 and 19^{th} element e19, for example, are not used in either transmission or reception in the transmission area. In such a case, the high-voltage switches 5 corresponding to the 17^{th} element e17 and 19^{th} element e19, for example, are desirably turned off. In the ultrasonic probe, cross-talk occurs between elements. As a result, a faint ultrasonic signal is transmitted at a channel corresponding to an adjacent element. The amount of the transmitted ultrasonic signal depends upon an electric impedance value, and if the value is low, the ultrasonic signal is transmitted dampened. As a result, the amount of the signal is reduced. Accordingly, the high-voltage switches are turned off, and channels interposed between transmitting channels can be used.

(Variation)

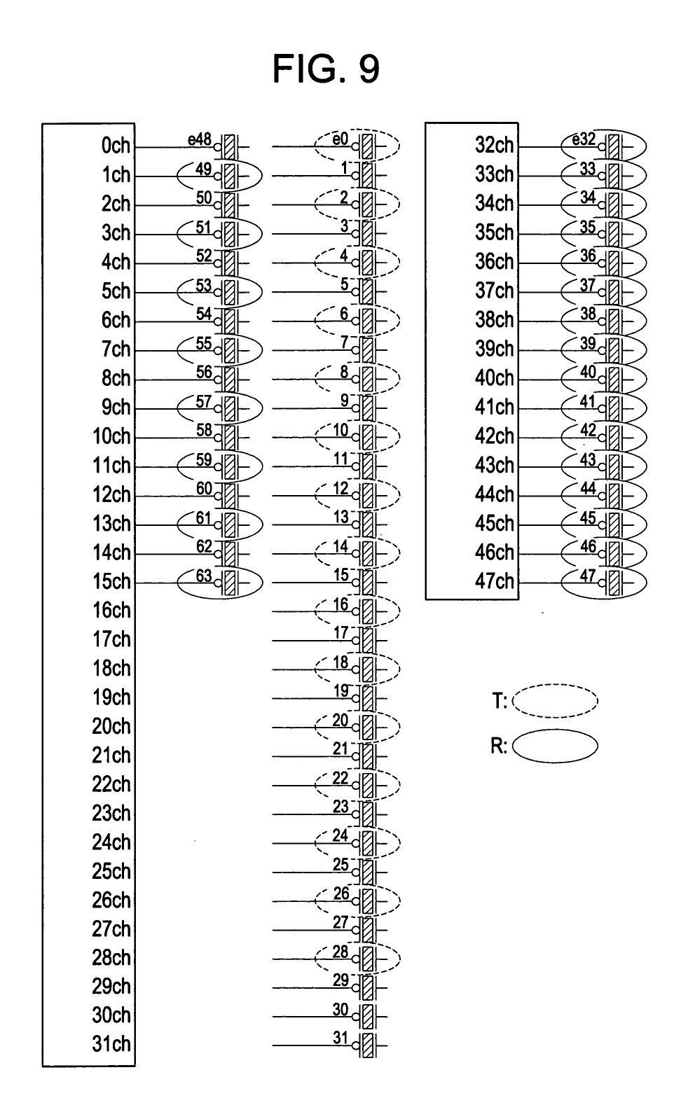

FIG. 9 is a schematic diagram generally showing another example of connections between channels and elements.

As shown in FIG. 9, in zero-th through 28^{th} elements e0-e28, every second element is connected to a transmitting channel via a high-voltage switch 5 (not shown). As compared with the connections shown in FIG. 7, the aperture in the transmission area is widened.

On the other hand, 32^{nd} through 63^{rd} elements e32-e63 outside the transmission area are connected to all channels other than those connected to these elements, and are together regarded as the reception area.

The high-voltage switches 5 corresponding to elements between the transmission and reception areas, i.e., 29^{th} through 31^{st} elements e29-e31, are turned on, and are connected to channels that conduct neither transmission nor reception.

Moreover, the high-voltage switches 5 corresponding to elements that are not used in either transmission or reception in the transmission area, such as 17^{th} element e17 and 19^{th} element e19, are turned off.

According to the present embodiment, a transmission area and a reception area are defined, and all channels other than those connected to transmitting elements are connected to the elements in the reception area and are used for reception. As a result, many channels can be used in reception to improve reception sensitivity.

Moreover, high-voltage switches 5 corresponding to elements between the transmission and reception areas are turned on to thereby electrically and mechanically reduce cross-talk from transmission to reception. On the other hand, high-voltage switches 5 corresponding to elements that are not used in either transmission or reception in the transmission area are turned off, and cross-talk generated between the transmitting channels can be used to transmit ultrasonic signals from the corresponding elements without driving the channels.

Furthermore, since many receiving channels can be provided as described above, it is possible to widen the aperture in the transmission area as in this variation, thus improving resolution of an image to be produced.

The ultrasonic diagnostic apparatus of the present invention is not limited to the aforementioned embodiments.

or example, in the ultrasonic diagnostic apparatus of the present invention, a matrix array probe in which transducers are arranged in a two-dimensional manner may be used in place of the sector probe. Moreover, the number of the channels in the transceiver section 6 and the number of elements in the ultrasonic probe 2 are illustrated by way of example, and they may be changed as needed. Besides, several modifications may be made without departing from spirits and scope of the present invention.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus for continuously transmitting ultrasonic signals toward a subject, continuously receiving signals reflected from said subject in response to the transmitted ultrasonic signals, and determining dynamics of blood flow in said subject based on the received signals, said apparatus comprising:
a transmitting/receiving device for continuously transmitting/receiving ultrasonic signals via a plurality of channels; and
a sector probe having transducers connectable to said channels via switches, said transducers being in a number larger than the number of said channels and arranged in one direction,
in which probe, ones among said transducers arranged in one direction that are spaced at intervals of a predetermined number of said transducers are connected to said channels for transmitting said ultrasonic signals.

2. The ultrasonic diagnostic apparatus of claim 1, wherein:
in said probe, every second said transducer is connected to one of said channels for transmitting said ultrasonic signals.

3. The ultrasonic diagnostic apparatus of claim 1, wherein:
in said probe, every third said transducer is connected to one of said channels for transmitting said ultrasonic signals.

4. The ultrasonic diagnostic apparatus of claim 1, wherein:
in said probe, said transducers are randomly connected to said channels for transmitting said ultrasonic signals in a predefined area.

5. The ultrasonic diagnostic apparatus of claim 1, wherein:
in said probe, said transducers whose corresponding switches are turned off are disposed between said transducers for transmitting said ultrasonic signals.

6. The ultrasonic diagnostic apparatus of claim 1, wherein:
in said probe, a transmission area in which said transducers for transmitting said ultrasonic signals are disposed from one end to the other end includes ¼-½ of all said transducers.

7. The ultrasonic diagnostic apparatus of claim 1, wherein:
in said probe, said transducers outside the transmission area in which said transducers for transmitting said ultrasonic signals are disposed from one end to the other end are used for receiving said ultrasonic signals.

8. The ultrasonic diagnostic apparatus of claim 7, wherein:
in said probe, said transducers that are not used in either transmission or reception of said ultrasonic signals are disposed between said transmission area and a reception area in which said transducers for receiving said ultrasonic signals are disposed.

9. The ultrasonic diagnostic apparatus of claim 8, wherein:
in said probe, said transducers disposed between said transmission area and said reception area are connected to said transmitting/receiving device via said switches.

10. A driving method for an ultrasonic diagnostic apparatus comprising a transmitting/receiving device for continuously transmitting/receiving ultrasonic signals via channels, and a sector probe having transducers connectable to said channels via switches, said transducers being in a number larger than the number of said channels and arranged in one direction, the method comprising the steps of:
continuously transmitting ultrasonic signals toward a subject while connecting said transducers to said channels in said transmitting/receiving device, and continuously receiving reflected signals while connecting said transducers in the other area to said channels; and
producing an image of said subject based on the received signals,
wherein said transmitting/receiving step comprises selecting a plurality of transducers in the predefined area spaced at intervals of a predetermined number of transducers, and transmitting ultrasonic signals while connecting said selected transducers to said channels.

11. The driving method of claim 10, wherein:
said transmitting/receiving step comprises transmitting said ultrasonic signals while selecting every second said transducer in the predefined area.

12. The driving method of claim 10, wherein:
said transmitting/receiving step comprises transmitting said ultrasonic signals while selecting every third said transducer in the predefined area.

13. The driving method of claim 10, wherein:
said transmitting/receiving step comprises transmitting said ultrasonic signals while randomly selecting said transducers in the predefined area.

14. The driving method of claim 10, wherein:
said transmitting/receiving step comprises disposing said transducers whose corresponding switches are turned off and which are not connected to any said channel between said transducers for transmitting said ultrasonic signals.

15. The driving method of claim 10, wherein:
in said transmitting/receiving step, the predefined area in which said transducers for transmitting said ultrasonic signals are disposed corresponds to ¼-½ of the area of all said transducers.

16. The driving method of claim 10, wherein:
said transmitting/receiving step comprises receiving said ultrasonic signals by said transducers outside the predefined area in which said transducers for transmitting said ultrasonic signals are disposed.

17. The driving method of claim 10, wherein:
said transmitting/receiving step comprises disposing said transducers that are not used in either transmission or reception of said ultrasonic signals between said transducers for transmitting said ultrasonic signals and said transducers for receiving said ultrasonic signals.

18. The driving method of claim 17, wherein:
said transmitting/receiving step comprises connecting said transducers disposed between said transducers for transmission and said transducers for reception to said transmitting/receiving device via said switches.
